(19) United States
(12) Patent Application Publication
(10) Pub. No.: US 2022/0013231 A1
(43) Pub. Date: Jan. 13, 2022

(54) METHOD FOR AI APPLICATIONS IN MRI SIMULATION

(71) Applicant: Corsmed AB, Stockholm (SE)

(72) Inventors: Christos Xanthis, Athens (GR); Anthony Aletras, Thessaloniki (GR)

(21) Appl. No.: 16/927,930

(22) Filed: Jul. 13, 2020

(51) Int. Cl. G16H 50/20; G01R 33/54; G01R 33/56; G06N 20/00; G16H 30/40; G16H 50/50; G06T 7/00; G06T 11/00; G06T 1/20

(52) U.S. Cl. CPC G16H 50/20; G01R 33/546; G01R 33/5608; G06N 20/00; G16H 30/40; G06T 2207/10088; G06T 7/0012; G06T 11/003; G06T 1/20; G06T 2207/20081; G16H 50/50

(57) ABSTRACT

The present invention describes a method for generation of training datasets for artificial intelligence (AI) applications in MRI (Magnetic Resonance Imaging), said method comprising
providing an MRI simulator;
providing input to the MRI simulator, said input being in the form of a pulse sequence and a computer-based anatomical model;
executing the MRI simulator and thus producing a simulated artificial MR image;
repeating the same procedure with different pulse sequences and/or the same pulse sequence, wherein when using the same pulse sequence then amending the characteristics of the same pulse sequence and/or amending the characteristics of the MR simulation when executing the MRI simulator;
   optionally also by amending the characteristics of the anatomical model;
   optionally also by amending the position and/or orientation of a plane/volume of interest of the anatomical model;
   optionally also by amending the characteristics of the MR simulation when executing the MRI simulator;
obtaining produced MR images;
producing a label map for each MR image; and
obtaining a training dataset based on all obtained produced MR images and/or label maps.

### FIELD OF THE INVENTION

The present invention relates to a method for generation of training datasets for artificial intelligence applications in magnetic resonance imaging.

### SUMMARY OF THE INVENTION

The present invention is directed to a method for generation of training datasets for artificial intelligence (AI) applications in MRI (Magnetic Resonance Imaging), said method comprising
providing an MRI simulator;
providing input to the MRI simulator, said input being in the form of a pulse sequence and a computer-based anatomical model;
executing the MRI simulator and thus producing a simulated artificial MR image;
repeating the same procedure with different pulse sequences and/or the same pulse sequence, wherein when using the same pulse sequence then amending the characteristics of the same pulse sequence and/or amending the characteristics of the MR simulation when executing the MRI simulator;
   optionally also by amending the characteristics of the anatomical model;
   optionally also by amending the position and/or orientation of a plane/volume of interest of the anatomical model;
   optionally also by amending the characteristics of the MR simulation/experiment when executing the MRI simulator;
obtaining produced MR images;
producing a label map for each MR image; and
obtaining a training dataset based on all obtained produced MR images and/or label maps.

As should be clear from above, the present invention is directed to AI applications for MRI. In particular, the method according to the present invention relates to using simulated MR images, i.e. artificial images, for training AI, i.e. obtaining a training dataset.

Furthermore, the method according to the present invention involves a repetition step in which something is changed in relation to the pulse sequence and/or the anatomical model and/or the characteristics of the MR simulation used in the first simulation. Either a different pulse sequence is used, or the characteristics of the same pulse sequence is altered. Another option is of course to both use a different pulse sequence and also amend the characteristics of this pulse sequence when comparing with the characteristics or design of the first pulse sequence used.

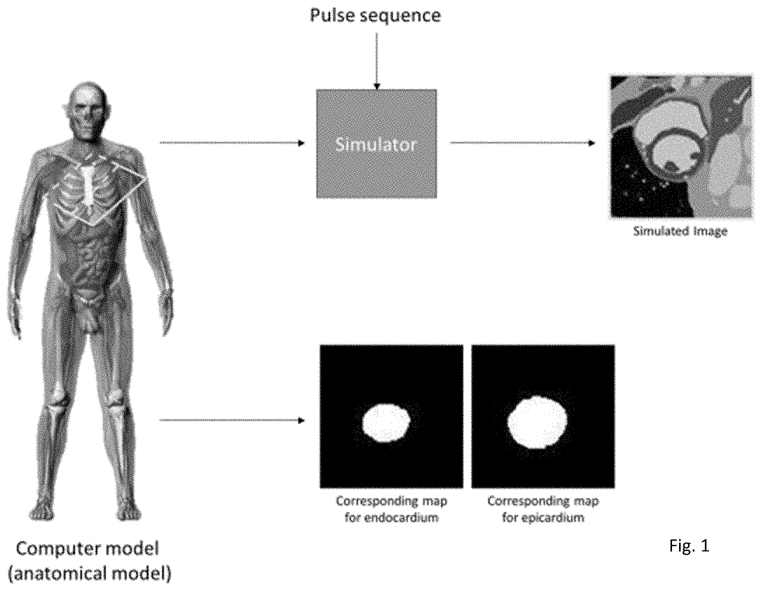

Moreover, in relation to the step of producing a label map, the expression “label map” may also be stated as “annotation map”. An example of a label map is shown in FIG. 1. In this example, the label map is of the same size as the simulated image and tells you if a pixel of the simulated image belongs to a tissue type (or tissue area) or not. It should be noted that it may also be of a different type, such as a label that says that in this image there is a tissue type (for example a tumor) or not. Depending on the application, it may have different form.

Specific Embodiments of the Invention

Below some specific embodiments of the present invention are disclosed and explained further.

According to one specific embodiment of the present invention, the step of obtaining all produced MR images and producing a label map for each MR image are both performed in the MRI simulator. In relation to this it should be noted that according to the present invention, the “MRI simulator” is a software. This further implies that the step of producing a label map for each MR image may be produced by this software, but this does not imply that MR simulation is performed in this step.

Moreover, according to another embodiment of the present invention, the steps of obtaining produced MR images and producing a label map for each MR image are performed in connection to each other, preferably simultaneously or alternately. Suitably, once the position of the slice-of-interest in 3D space is defined, then the production of images and label maps can run in parallel.

As mentioned above, the method according to the present invention involves repetition by using a different pulse sequence or amending the characteristics of the same pulse sequence, or both. Moreover, according to one specific embodiment, the characteristics of the anatomical model is also amended. According to yet another embodiment, the position and/or orientation of a plane/volume of interest of the anatomical model is also amended.

According to yet another embodiment, the method involves amending the characteristics of the MR simulation/experiment when executing the MRI simulator. Non-limiting examples of parameters are the BO inhomogeneity, noise, artefacts, etc.

According to yet another embodiment, the method involves repeating the same procedure with different pulse sequences and/or the same pulse sequence, amending the characteristics of the anatomical model, and amending the position and/or orientation of a plane/volume of interest of the anatomical model.

As hinted above, the MRI simulator according to the present invention suitably is a software. According to one specific embodiment, the MRI simulator is web-based and cloud-based. This has advantages both for the user as such and in relation to data handling, transfer and storage.

Furthermore, according to yet another implementation embodiment of the present invention, the method involves simulation of a magnetic resonance (MR) scanner in the MRI simulator, said method comprising

input of data parameters into a web interface of the MRI simulator;

connection of the web interface with a cloud-based simulator engine of the MRI simulator for transfer of data parameters to the cloud-based simulator engine;

recalculation of the data parameters for the provision of one or more simulated MR signals, said recalculation being performed in the cloud;

reconstruction of an MR image based on said one or more simulated MR signals, said reconstruction of an MR image being performed in the cloud; and

sending the MR image to the web interface.

In relation to the web interface of the MRI simulator it should be noted that a corresponding web-service may be used instead of a specific web interface. Furthermore, in relation to the last step of sending the MR image to the web interface it should be noted that this step may also be performed instead by calling a web-service.

Furthermore, according to one specific embodiment, the cloud-based simulator engine performs the recalculation and sends recalculated data to one or more GPUs (graphics processing units) of the MRI simulator, which GPUs sends back said one or more simulated MR signals. Furthermore, according to yet another embodiment, the step of reconstruction of an MR image is performed by one or more CPUs (central processing units) and/or one or more GPUs (graphics processing units) of the MRI simulator in the cloud.

### DESCRIPTION OF THE DRAWINGS AND FURTHER EXPLANATION

In FIG. 1 there is shown one schematic implementation for the method according to the present invention. The simulator accepts two inputs: the pulse sequence and the computer (anatomical model). For every execution of the simulator, a simulated (artificial) MR image is produced. The suggested methodology provides a flexible solution that allows the generation of training datasets for multiple variations of both the imaging protocol and the computer model, which is not easy today with a real MRI experiment configuration (MRI system, patient recruitment, etc.). In addition, the digitized and highly-customizable nature of the anatomical model allows for the concurrent production of well-annotated data in the form of tissue masks. The annotation of the artificial data is always objective and depicts the real tissue characteristics without being affected by various factors that may deteriorate the quality of the medical image, such as noise, limited spatial and temporal resolution due to the pulse sequence design, etc. Variants where noise and the presence of other artefacts that can make the data look close to real can easily be accommodated.

As explained above, the method according to the present invention involves repeating the same process where conditions are altered. The repetition is performed for different:
Pulse sequences or configuration of the same pulse sequence
Characteristics of the anatomical model
Position and/or orientation of the plane/volume of interest
Characteristics of the MR experiment such as the BO inhomogeneity, noise, artefacts, etc.

A set of hundreds of artificial MR images and the corresponding map(s) are produced, and they are used as a training dataset for training a neural network. The neural network is then tested on true MR images (images from patients and volunteers).

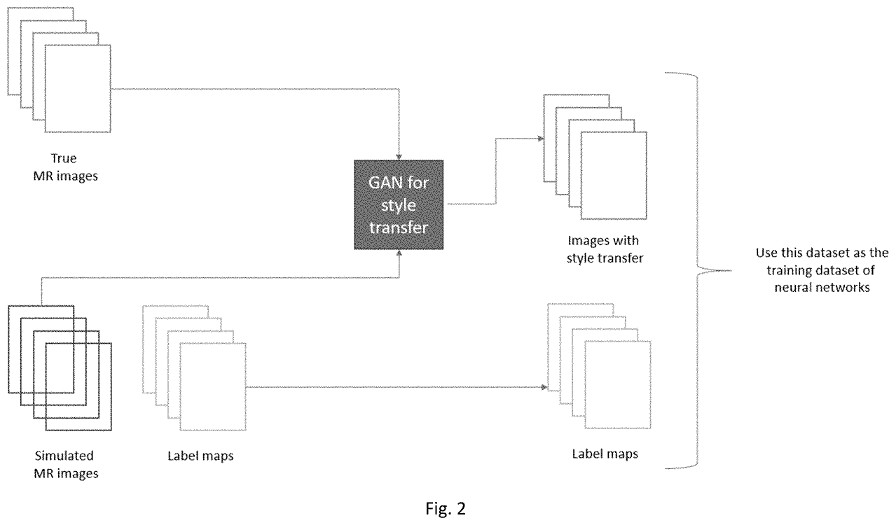

In FIG. 2 there is shown one extension of the method according to the present invention. True MR images are acquired from a patient in the MR scanner. Simulated MR images and the corresponding label maps are produced by the MR simulator. A Generative Adversarial Networks (GAN) style transfer is utilized in order to take two images (true and simulated) and this style is applied from one image to the other image. The new image (green on the figure) looks more realistic than the simulated one since it keeps the style of the true image. Moreover, the new image (green again) comes with the corresponding label map since it keeps the structure of the simulated image. It is expected that the resulting dataset is more representative and will improve the training of neural networks.

1. A method for generation of training datasets for artificial intelligence (AI) applications in MRI (Magnetic Resonance Imaging), said method comprising
providing an MRI simulator;
providing input to the MRI simulator, said input being in the form of a pulse sequence and a computer-based anatomical model;
executing the MRI simulator and thus producing a simulated artificial MR image;
repeating the same procedure with different pulse sequences and/or the same pulse sequence, wherein when using the same pulse sequence then amending the characteristics of the same pulse sequence and/or amending the characteristics of the MR simulation when executing the MRI simulator;
optionally also by amending the characteristics of the anatomical model;
optionally also by amending the position and/or orientation of a plane/volume of interest of the anatomical model;
optionally also by amending the characteristics of the MR simulation when executing the MRI simulator;
obtaining produced MR images;
producing a label map for each MR image; and
obtaining a training dataset based on all obtained produced MR images and/or label maps.

2. The method according to claim 1, wherein the step of obtaining all produced MR images and producing a label map for each MR image are both performed in the MRI simulator.

3. The method according to claim 1, wherein the steps of obtaining produced MR images and producing a label map for each MR image are performed in connection to each other, preferably simultaneously or alternately.

4. The method according to claim 1, wherein the characteristics of the anatomical model is also amended.

5. The method according to claim 1, wherein the position and/or orientation of a plane/volume of interest of the anatomical model is also amended.

6. The method according to claim 1, wherein the method involves amending the characteristics of the MR simulation when executing the MRI simulator.

7. The method according to claim 1, wherein the method involves repeating the same procedure with different pulse sequences and/or the same pulse sequence, amending the characteristics of the anatomical model, and amending the position and/or orientation of a plane/volume of interest of the anatomical model.

8. The method according to claim 1, wherein the MRI simulator is web-based and cloud-based.

9. The method according to claim 8, wherein the method involves simulation of a magnetic resonance (MR) scanner in the MRI simulator, said method comprising
input of data parameters into a web interface of the MRI simulator;
connection of the web interface with a cloud-based simulator engine of the MRI simulator for transfer of data parameters to the cloud-based simulator engine;
recalculation of the data parameters for the provision of one or more simulated MR signals, said recalculation being performed in the cloud;
reconstruction of an MR image based on said one or more simulated MR signals, said reconstruction of an MR image being performed in the cloud; and
sending the MR image to the web interface.

10. The method according to claim 9, wherein the cloud-based simulator engine performs the recalculation and sends recalculated data to one or more GPUs (graphics processing units) of the MRI simulator, which GPUs sends back said one or more simulated MR signals.

11. The method according to claim 9, wherein the step of reconstruction of an MR image is performed by one or more CPUs (central processing units) and/or one or more GPUs (graphics processing units) of the MRI simulator in the cloud.
